(19) United States
(12) Patent Application Publication
(10) Pub. No.: US 2010/0310686 A1
(43) Pub. Date: Dec. 9, 2010

(54) EXTRACTS OF DESCHAMPSIA ANTARCTICA DESV, WITH ANTINEOPLASTIC ACTIVITY

(75) Inventors: Manuel Gidegel, Santiago (CL); Helga Weber, Pucon (CL); Gustavo Cabrera, Temuco (CL); Ana Gutierrez, Temuco (CL); Jennifer Osorio, Temuco (CL); Jose Becera, Concepcion (CL); Osvaldo Podhajcer, Buenos Aires (AR); Eduardo Cafferata, Buenos Aires (AR); Carlos Sunkel, Madrid (ES); Ivan Mihovilovic, Miami, FL (US)

Correspondence Address: John Dodds, 1707 N St NW, Washington, DC, 20036, US

(73) Assignee: Uxmal S.A., Chile, Santiago (CL)

(21) Appl. No.: 12/734,597

(22) PCT Filed: Nov. 14, 2008
(86) PCT No.: PCT/US2008/012819

§ 371 Date: May 11, 2010

Related U.S. Application Data
(60) Provisional application No. 61/003,058, filed on Nov. 14, 2007.

(51) Int. Cl. A61K 36/899

(52) U.S. Cl. 424/750

(57) ABSTRACT

The present disclosure provides a novel antineoplastic extract obtained from Deschampsia antarctica plant. Active components of the antineoplastic extract are also disclosed and a method to prevent proliferation of cancerous or tumorous cells with the extract or with components thereof is disclosed. Furthermore, a method to induce production of active ingredients in in vitro grown plants by submitting the plants to physical or chemical treatements before preparing the antineoplastic extract is disclosed. This disclosure provides compostions of tablets and pellets for treating patients with cancer or for prevention of occurence of cancerous diseases.

### PRIORITY

This application claims priority of the U.S. provisional application No. 61/003,058 filed on Nov. 14, 2007.

### FIELD OF THE INVENTION

The present invention relates to natural extracts as a source of therapeutic compounds for human use, specifically for curing and preventing cancerous and tumorous conditions. More specifically, the present invention relates to extracts, composition of the extracts and methods to produce the extracts from Deschampsia antarctica for prevention of cancer.

### DESCRIPTION OF RELATED ART

Cancer is the second leading cause of death in developed countries. Due to the parallel increase in the incidence of this disease as compared to the increase in the life expectancy of the population, there is a great medical and social interest in this pathology. The five types of cancer most common in the world are lung cancer, stomach cancer, breast cancer, colorectal cancer and uterine cancer.

The incidence and mortality from cancer have increased in many countries. According to the World Health Organization (WHO), more than 10 million people suffer cancer each year. That number is expected to increase by 2.4% annually, to 15 million people per year by 2020.

In the specific case of colorectal cancer (CRC), each year approximately one million new cases occur in the world and half a million deaths, the world mortality rate being 8.1/100,000 inhabitants. This type of cancer is largely seen in the more developed regions (25.1/100,000 inhabitants), representing the second leading cause of death from cancer in Europe and in the United States. More than 130,000 new cases are diagnosed annually in the United States alone and more than 370,000 new cases in Europe. In Argentina, the figures for new cases are similar to those occurring in the United States.

In Chile, cancer is the second leading cause of death. Intestinal cancer is responsible for 46.2% of the deaths from this pathology. Among that number, colorectal cancer is the third leading cause of death and it is constantly increasing in frequency. The mortality from this disease presented a significant rising trend in the period 1990-2003.

Close to 70% of patients who suffer from colorectal carcinoma must undergo a surgical resection while 30% to 40% have a relapse. The liver is the most frequent site of CRC metastasis and a complete resection of the hepatic metastasis is the only alternative cure. However, surgery is possible for only 20% of patients at the time of diagnosis and the average survival rate at 5 years is from 25% to 40%, even with chemotherapy.

The actual CRC treatment is ineffective in the advanced stages of the disease. An average survival rate of patients with metastatic colorectal carcinoma who receive state-of-the-art medication, like Erbitux and Avastin, as part of the first line of treatment is only 15 to 20.5 months. This demonstrates the need to look for more effective therapies to increase the patient's chances of survival.

On the other hand, colonoscopy is the only screening that allows the detection and removal of pre-malignant lesions. However, this test is very complex, in terms of patient preparation and inconvenience and patient discomfort. Therefore, CRC diagnosis at a relatively early stage is a rare event and only 9% of patients are detected at stage 1 of the disease when the possibilities of cure are larger. Therefore, the possibility of preventing CRC development by using nutraceutical compounds is of high importance.

On the other hand, Deschampsia antarctica Desv. is one of the two phanerogamous plants that have successfully colonized the Antarctic. It is found on several ocean islands in the south and it is restricted in the Antarctic territory to the Antarctic peninsula and islands offshore. This taxon has adapted to survive in hostile conditions. Among the characteristics that might be involved in the survival of this grass in the adverse Antarctic environment are a tolerance to extra cellular ice, a photosynthetic apparatus that maintains 30% of the photosynthetic optimum at 0° C., and the accumulation of carbohydrates as fructans and saccharose. The Antarctic territory is an adverse zone for the development of vascular plants. D. antarctica only grows in the summer season when photosynthetically active radiation (PAR) can reach 2000 μmol m⁻²s⁻¹ and the temperature is usually from −2 to 5° C.

Research on natural products as a source of therapeutic compounds for human use reached its peak in the 80's. 49% of the 877 small molecules introduced on the market between 1981 and 2002 came from natural products or their synthetic derivatives (Newman, D J; J. Nat. Prod. 66: 1002-1037 (2003)). Despite this, pharmaceutical research on natural products has experienced a slight drop in the last decade. This was essentially due to the search for new compounds based on the development of combinatorial libraries and progress in molecular biology that have led to the design of “smart” chemical molecules like Iressa/Imanitib, aimed to target Epidermal Growth Factor Receptors. However, the experience in recent years has revived the interest of pharmaceutical companies in compounds derived from natural products, since it has been demonstrated that they have a greater number of chiral centers and steric complexity than any synthetic product or recombinant library (Free M. J. Chem. Inf. Comput. Sci. 43: 218-227 (2003)). The main reason for this failure is that recombinant libraries essentially look for compounds based on chemical accessibility but they have restrictions in terms of increasing chemical diversity (Martin Y. C. J. Comb. Chem. 1:32-45 (1999)).

There are publications suggesting beneficial effects of fruit- and vegetable consumption in lowering the risk of various cancers, including colorectal cancer. Kaur et al. provide data showing chemopreventive effects of grape seed fruits (Kaur M, Clin Cancer Res 12(20): 6194-6202 (2006)). Lu et al. show data suggesting chemopreventing effects of green tea on lung cancer (Lu Y, Cancer Res. 66 (4): 1956-1963 (2006)). Suggestions of natural ingredients having chemopreventive effects along with the failure to meet the expectations regarding combinatorial chemistry have revived a pharmaceutical interest in natural compounds.

Accordingly, there is a constant need for natural compounds and extracts that could help preventing and curing the commonly occurring cancer diseases.

### SUMMARY OF THE INVENTION

This invention comprises natural extracts containing compounds with antineoplastic activity that will help to cure and prevent diseases that have a high incidence among the population. In particular, a natural product is described, said product being extracted from an organism adapted to survive the high radiation on the Antarctic Continent.

This invention provides a novel antineoplastic extract and a method to obtain the extract from Deschampsia antarctica.

This invention provides a natural antineoplastic extract to prevent proliferation of cancer cells.

Furthermore, this inventions provides active composites of the natural antineoplastic extract.

This invention also provides compositions for treating patients with existing cancer condition.

Moreover, this invention provides a method to increase the amount of antineoplastic compounds in Deschampsia antarctica tissue, and accordingly a method to extract the compounds of the plant tissue.

Even further, this invention provides a method to obtain an antineoplastic extract from in vitro grown Deschampsia antarctica plants.

Still further, this invention provides a method to prepare in vitro cultivated Deschampsia antarctica plants for material of antineoplastic preparations.

Accordingly, this invention also provides antineoplastic preparations comprising Deschampsia antarctica extract or components of the extract.

### BRIEF DESCRIPTION OF THE FIGURES

FIG. 1. UV-Vis 200-400 nm spectra diagram. A) Plants of Deschampsia antarctica collected in vivo. B) Plants of Deschampsia antarctica cultivated in vitro without any treatments (Control).

FIG. 2. UV-Vis 200-400 nm spectra diagram of plants of Deschampsia antarctica (in vitro) treated with NaCl solution. A) 2 M NaCl, B) 3 M NaCl, and C) 4 M NaCl.

FIG. 3. UV-Vis 200-400 nm spectra diagram of plants of Deschampsia antarctica (in vitro) treated with UV radiation. A) 45 μW/cm², B) 70 μW/cm².

FIG. 4. Effects of extract fractions of Deschampsia antarctica in concentrations of 100 μg/ml on in vitro growth of colon cancer cells (HT29 and LoVo), hepatic cancer cells (Hep3B) and control cells (wi38). GCB1, GCB2 and GCB3 correspond to extractions of three different collections of Deschampsia antarctica plants from the Antarctic. The extracts were prepared with water, then dried and dissolved in methanol. Control contains no extract added and the methanol control is a control containing 5% methanol as was used to dissolve the dried aqueous extract.

FIG. 5. Effects of ethyl acetate (a and b) and methanol (c and d) extracts at a concentration of 75 ug/ml on LoVo colorectral cancer cells (a and c) and wi38 human fetal fibroblast cells (b and d).

FIG. 6. Effects of luteolin derivatives (peak 1 and peak 2) obtained from an extract of Deschampsia antaractica plants at different concentrations (0.017, 0.17 and 1.7 mM) on cellular viability of colorectal cancer cells LoVo. a) shows effects of Peak 2 derivative and b) shows effect of a combination of peak 1 and peak 2 derivatives.

FIG. 7. Semipreparative HPLC analysis of Deschampsia antarctica extract.

FIG. 8. HPLC chromatogram of the main compounds of Deschamcpisa antarcitca extracts. A) peak 1 and B) peak 2.

FIG. 9. UV-visible spectra of the main compounds of Deschampsia antarctica extracts a) peak 1 spectrum and b) peak 2 spectrum.

FIG. 10. Mass spectra of a) peak 1, and b) peak 2 isolated from Deschampisa antarctica extract.

FIG. 11. Chemical structure of A) peak 1 compound and B) peak 2 compound.

FIG. 12. Chemical structure of orientin.

### DETAILED DESCRIPTION OF THE INVENTION

Deschampsia antarctica Desv. (Poacea) is one of the two vascular plant species that have naturally colonized Maritime Antarctic Peninsula. During the recent years, D. antarctica has experienced an increasing exposure to ultraviolet radiation, due fundamentally to the hole in the ozone layer present in the Antarctic Region. Consequently, this plant species has modified its metabolism to increase the production of secondary metabolites that intervene in the photoprotection process.

The fact that D. antarctica is naturally acclimated to conditions that expose it to oxidative stress (high light and low temperature) let us to consider this plant as a source of antioxidative compounds. It was possible to obtain antioxidative compounds from the plants that grew in wild in Antarctica but the feature was practically lost when the plants were cultivated in vitro. This disclosure provides methods to induce antioxidative compounds in in vitro grown plants and further more the invention according to this disclosure provides an antineoplastic extract obtained from the plants.

This invention authentically establishes the antitumorigenic effect of the extracts obtained from D. antarctica and their capacity to prevent the disease through the study of their in vitro effects.

This invention also describes a method to induce antineoplastic compounds in in vitro grown plants and isolation of the compounds, as well as their potential applications.

The products according to this invention are based on the metabolites with antineoplastic activity present in D. antarctica.

The invention is described below by means of examples. The examples are not meant to limit the scope of the invention.

### EXAMPLE 1

### Comparison of the Absorption Peaks of Extract From Naturally Grown Plants to the Extract of In Vitro Grown Plants Without Stress Induction

Deschampsia antarctica material was collected from Robert Island, a copper mine peninsula (62°22′S; 59°43′W), and it was carried in plastic bags. The material was disinfected with fungicide (Benomyl and Captan) and sodium hypochlorite. Plant material was micropropagated in vitro. The culture medium was prepared based on the Murashige and Skoog (MS) medium. 1 mg/l of BAP hormone (N6 Benzylaminopurine) was added as well as 35 mg/l saccharose and 9 g/l of agar at a final pH of 5.7. The in vitro growing plants were kept in growth chambers at 22° C. with a photoperiod of 16/8 h (light/darkness) and a photon flow of 2000 μmol m⁻² s⁻¹.

The aerial parts of the in vivo or in vitro growing plants were collected and macerated in 5 ml of distilled water. The maceration is later sonicated for 10 minutes and centrifuged at 1,000 rpm for 15 minutes

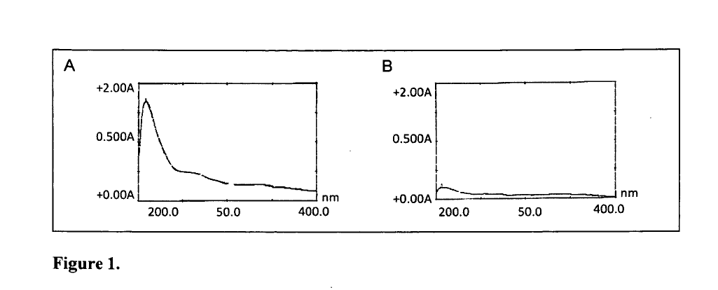

A thin layer chromatography was performed. The extracts were seeded on a 60 F₂₅₄ silica gel slide (MERCK) to visualize the compounds present. A UV-Vis SHIMADZU UV-160 spectrophotometric analysis was used to analyze the extracts. An absorbency screening was consequently conducted between 200 and 400 nm to determine the presence of absorption maximums characteristic of the families of compounds present in the extracts (FIG. 1).

When the flavonoids are dissolved in methanol, flavones and flavonols exhibit two major peaks of absorption in the 240-400 nm region when they are examined by UV spectroscopy and visible light. These peaks commonly refer to band I (300-400 nm) and band II (240-280 nm). According to the UV-Visible 200-400 nm spectrum analysis (FIG. 1), flavonoids are seen in the metabolic extracts of plants collected in vivo in the Antarctic. They are virtually absent in the extracts of plants cultivated in vitro in the laboratory. The flavonoids exhibit characteristic peaks.

### EXAMPLE 2

### Polyphenol Induction in the In Vitro Plants

As the previous example shows, flavonoids were virtually absent from the in vitro grown plants. This example shows that the flavonoid production in Deschampisa antarctica plants is inducible by various stress conditions.

Induction by Salt

After 50 days, the in vitro propagated Deschampsia antarctica plants were fully removed from the agar, and the roots were cleaned from all the agar.

After being removed from the agar, the plants were submerged in aqueous solutions of different concentrations of NaCl (2, 3 and 4 M) for a period of 30 minutes, after which the aerial part of the plant is macerated in 5 ml of distilled water. The maceration is later sonicated for 10 minutes and centrifuged at 1,000 rpm for 15 minutes.

Induction by Exposure to UV Radiation

The plantlets grown in agar were irradiated by UV light, in the same jar in which they grew, at intensities of 45 μW/cm² and 70 μW/cm² for 2 hours. Plantlets were then removed from the agar and the aerial part of the samples was cut off, macerated with 5 ml of methanol, sonicated for 10 minutes and centrifuged at 1000 rpm for 15 minutes. The extracted samples were concentrated, lyophilized and stored at −20° C.

Polyphenol Concentration in the Extract of In Vivo and In Vitro Grown Plants

Unlike Deschampsia antarctica plants cultivated in vitro, the plants cultivated in natural conditions showed continuous induction of polyphenols throughout their entire stage of development. The plants are exposed to constant risks of saline solutions with a concentration of 0.5 M (concentration in sea water) and to the conditions of the natural radiation in Antarctica.

The extracts obtained from the plants of Deschampsia antarctica cultivated in vitro and in in vivo were submitted to analysis through a UV-Vis 200-400 nm spectrum to determine the peak absorbencies of the compounds present in the extracts against methanol in a SHIMADZU UV-160 spectrophotometer. 100 μl of the extract (macerated) was used, specifically from the supernatant, and dissolved in 10 ml of methanol.

Deschampsia antarctica plants cultivated in vitro without any treatment were used as controls in order to evaluate the effects of the treatment

It can be clearly seen in the spectra that all stress treatments to which the plants were submitted caused an increase in the concentration of polyphenols as compared to the control (FIGS. 1-3).

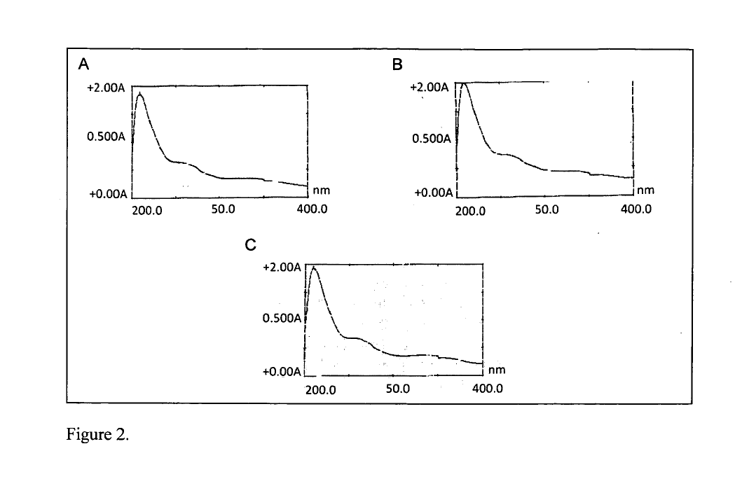

The treatment with NaCl solutions (FIG. 2) that caused the greatest increase in the concentration of polyphenols was observed when the plant was submerged in 3M NaCl, although NaCl at concentrations of 2 and 4 M exhibited a similar effect. The present data also revealed that 3M NaCl is the concentration at which Deschampsia antarctica exhibited the highest polyphenol concentration. The response of Deschampsia antarctica in terms of polyphenol production in the presence of different concentrations of NaCl is: 3M>4M>2 M.

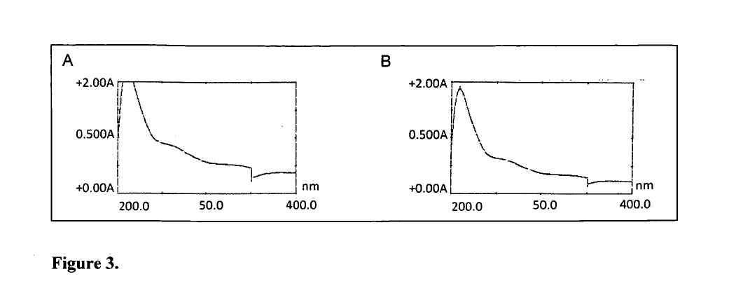

The plants that were exposed to 45 μW/cm² of UV radiation for 2 hours showed the greatest increase in polyphenols (FIG. 3). The plant exposed to greater radiation intensity (70 μW/cm²) during the same period of time did not reveal an increase as high as for 45 μW/cm². This may occur because the plants suffered some type of damage that caused the green matter to die or the plants spent resources on recovering from the damage, thereby diminishing the concentration of secondary metabolites.

A comparison of the results obtained with Deschampsia antarctica submitted to different treatments clearly reveals that the best result or the greatest increase in polyphenols was observed when the plants were submitted to UV radiation at an intensity of 45 μW/cm² for 2 hours (FIGS. 1, 2 and 3).

### EXAMPLE 3

### Fractioning of the Aqueous Extracts

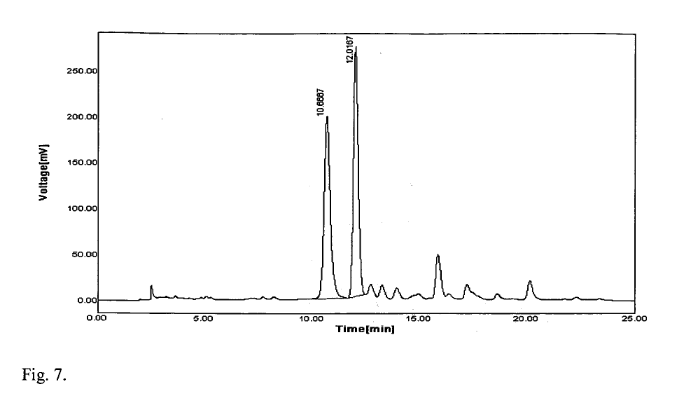

The aqueous extract of Deschampia antarctica was analyzed with HPCL analysis. FIG. 7 shows a chromatogram of the HPLC analysis. The chromatogram shows the main components of Deschampsia antarctica aqueous extract. Two major peaks (peak 1 with retention time of 10.6667 min and peak 2 with retention time of 12.0167 min) account to 80% (w/w) of the total amount of injected sample.

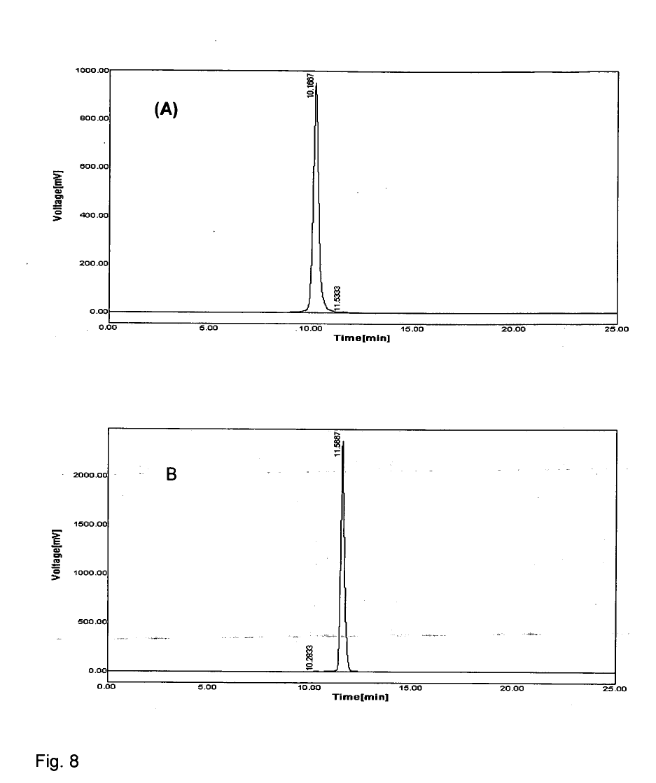

Both of the peaks were collected separately by using a semipreparative column. For purity assessment an HPLC-DAD equipped with an analytical column was used. The chromatograms corresponding to the isolated peaks are presented in FIG. 8. Each chromatogram shows only one peak with purity higher than 95% indicating that the peaks were essentially pure.

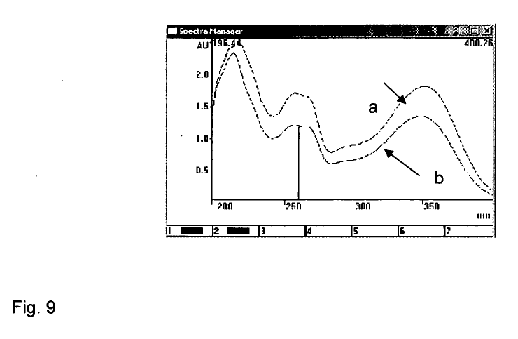

By using a Diode array analytical HPLC it was possible to obtain a UV-visible 200-400 nm spectrum for both of the compounds. The spectra are shown in FIG. 9.

Both spectrums showed major absorption bands in 240-400 nm region. Both exhibited first absorption band at 260 nm and second one at 350 nm. Those peaks are in close agreement with the absorption spectra exhibited by flavonoids when analyzed using UV-visible spectroscopy. When flavonoids are dissolved in methanol, flavones and flavonols show tow major peaks known as band I (300-400 nm) and band II (240-280 nm), respectively.

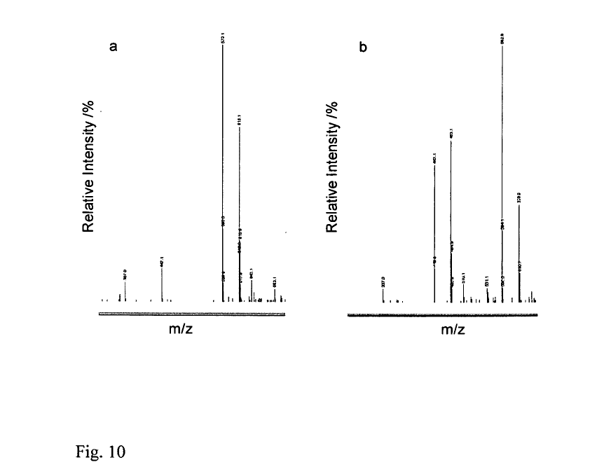

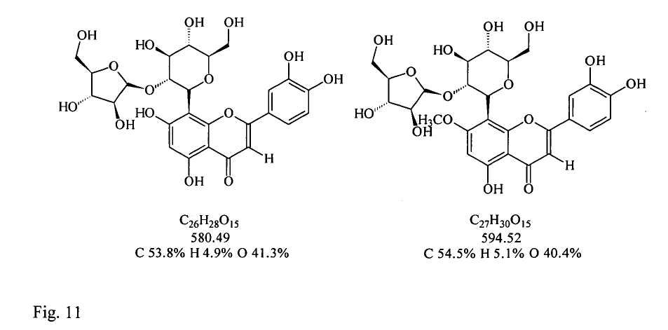

In order to elucidate the chemical structures of the compounds, we coupled HPLC to mass spectrometer to obtain the mass chromatogram of peaks 1 and 2 (FIG. 10). The mass spectra chromatogram showed the main peaks at m/z 580 for peak 1 and m/z 593 for peak 2. This information was compared with other mass spectra by using a mass spectra library for natural compounds and the resulting structures are presented in FIG. 11. Peak 1 corresponds to isoswertiajaponin((7-O-methylorientin) 2″-O-beta-arabinopyranoside) and peak 2 corresponds to orientin 2″-beta-arabinopyranoside. These compounds have been previously identified in Deschampisa antarctica leaves (Webby R. and Markham K, 1994, Isoswertijaponin 2″-O′beta-arabinopyranoisee and other flavone-C-glycosides from the Antarctic grass Deschampisa antarctica. Phytochemistry 36(5): 1323-1326). However, no biological activity of the identified compounds has been proposed. Thus, the present disclosure is the first report concerning the biological activity of these natural products.

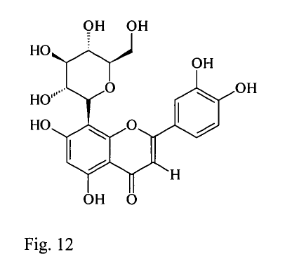

The mass spectra of both of the compounds showed a common fragment which appears at m/z 448 that belongs to orientin (FIG. 12). Several biological activities, such as radioprotection, vessel relaxation, antioxidant properties, free radical inhibitor properties and antiviral activity have been published for orientin.

### EXAMPLE 4

### In Vitro Inhibition of Malignant Cells Growth

Fractionation of Total Extracts

The total plant extracts were fractionated into compounds by paper chromatography. The sample was seeded on Whatman No. 3 paper using 15% glacial acetic acid as the mobile phase. The different compounds were visualized under UV light. The different fractions, called B1, B2 and B3, were recovered from the paper by immersion in methanol and then concentrated in a rotoevaporator. The slide chromatography was conducted to visualize the isolated compounds in each fraction

Chemical Structure of the Fraction With Biological Activity

According to the results provided by the HPLC-mass spectrometry (Example 3 above) it can be assumed that luteolin with different degrees of glycosylation and substitution of glycosides through C—C bonds (orientin compounds) is the molecule that is largely present and causing biological activity. This type of structure increases the stability of the active compound. Moreover, these compounds were present in extracts of in vivo grown Antarctic Deschampsia plants or plants subjected to 4° C. for 72 hours, but they are not present in plants produced in vitro at 13° C. (data not shown). This indicates that these compounds are inducible at low temperatures or other types of stress the plants experience in wild.

It is known that flavones play an important role in the human body as an antioxidant, chelators of free radicals, anti-inflammatory agents, promoters of the metabolism of carbohydrates and stimulators of the immune system (Rahman, I., Biswas, S. K., Kirkham, P. A. 2006. Regulation of inflammation and redox signaling by dietary polyphenols. Biochem Pharmocol. 702(11): 1439-1452; Kandaswami, C. Lee, L. T. Lee, P. P, Hwang J. J.; Ke. F. C., Huang, Y. T. Lee, M. T. 2005 In Vivo 19(5) 895-909). However, there is no research or indications of Deschampsia antactica extracts of being antineoplastic.

In order to determine whether the methanol extracts obtained from D. antarctica could have some antineoplastic effect, the soluble fractions B1, B2 and B3 were tested. These fractions were obtained from the total fraction and have different degrees of glucoside substitution.

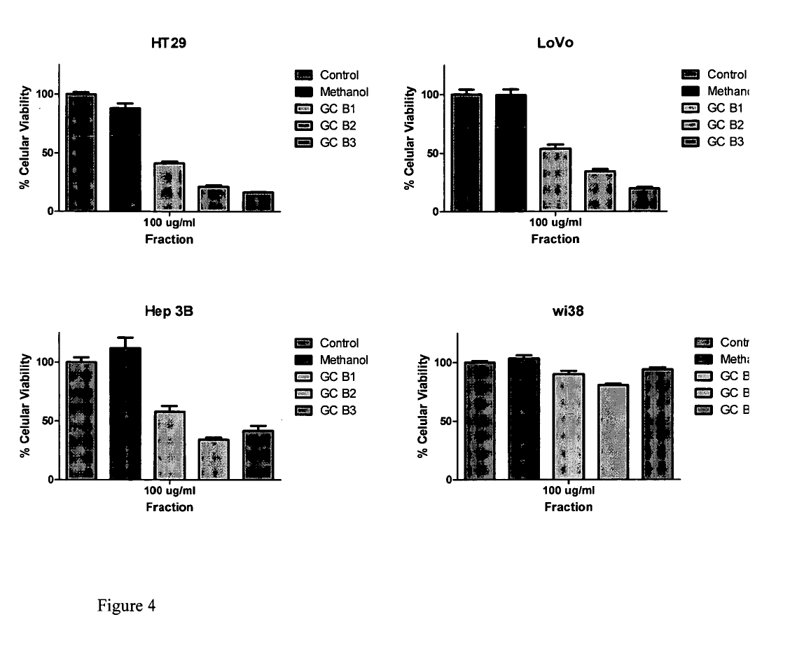

FIG. 4 shows the effect of the B1, B2 and B3 fractions on in vitro growth of colon cancer cells and hepatic cancer cells. It can be seen that these fractions effectively inhibit the proliferation of human HT 29 and LoVo colorectal cancer cells and Hep3B hepatoma cancer cells while the B3 fraction, with the highest degree of glucoside substitution, presents the greatest level of inhibition on malignant cellular proliferation (FIG. 4 HT29, LoVo and Hep 3B). Its effect on WI38 (normal lung fibroblasts) was tested at the maximum concentration to determine specificity and toxicity. No inhibitory effect on WI38 cells proliferation was observed (FIG. 4).

It can be concluded that these compounds can inhibit malignant cells growth in vitro, but showed no inhibitory effect on the proliferation of normal fibroblasts. This data indicate the antineoplastic effect of these fractions.

As shown in Examples above, the antineoplastic compounds extracted from Deschampsia antarctica could be induced in vitro. Therefore, the amount of antioxidants to be produced in plants by exposure to UV light, salt treatment or low temperature can be modulated. Moreover, the production of the antineoplastic extract becomes actually practicable as the plant material can be cultivated in large amounts in vitro.

Besides working with the soluble fractions mentioned before, Deschampsia antarctica plant material was extracted with solvents of increasing polarity (ethyl acetate and methanol). The aim of this approach was to divide plant constituents into fractions of different polarity on extraction. Organic solvent extracts were made in a Soxhlet apparatus.

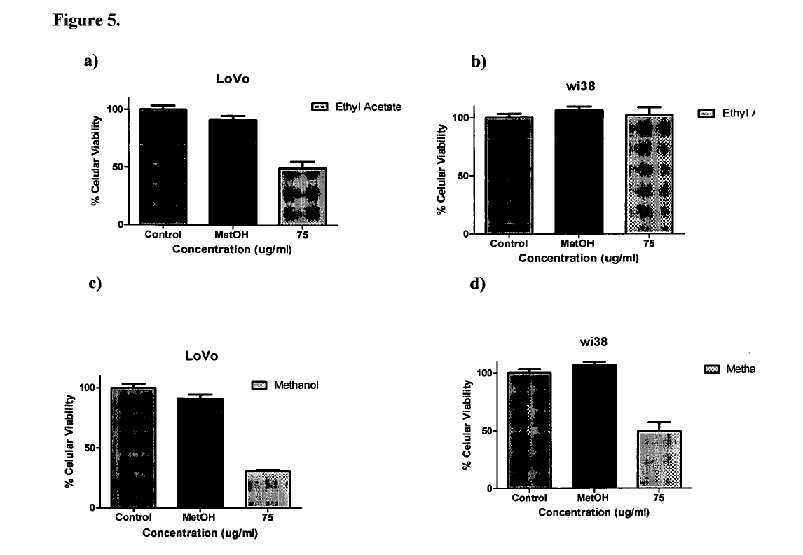

FIG. 5a shows the effect of an ethyl acetate extract on in vitro growth of human colon cancer cells (LoVo). An inhibition of 50% was observed in the cellular proliferation of these tumoral cells. The same extract was tested in WI 38 cells (normal lung fibroblasts). No inhibitory effect on WI 38 cells proliferation was observed (5b).

On the other hand, FIG. 5c shows the effect of a methanol extract, which produced more than 50% of inhibition on the proliferation of colon tumoral cells (LoVo). This extract was tested in non-tumoral cells (WI38), showing an inhibitory effect on cell proliferation (FIG. 5d). The methanol and ethyl acetic extracts were active against LoVo colorectal cancer cells at the lowest concentration of 75 ug/ml.

The most active fractions were used for further fractionation steps. This procedure led to the isolation of pure compounds (see Example 3 above). We also tested these pure compounds (peak 1 and peak 2 of example 3 above) and a combination of them on tumoral and non-tumoral cells.

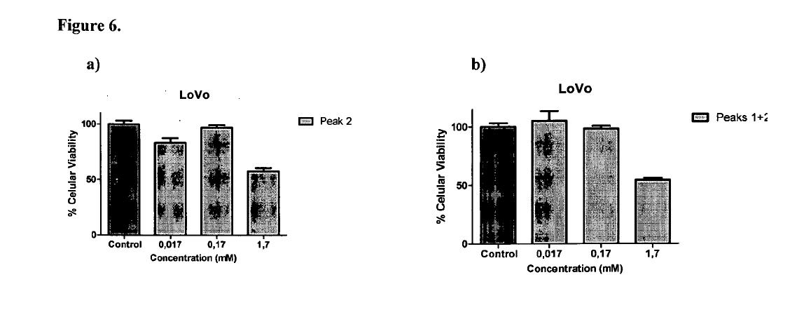

FIGS. 6a and 6b show the inhibitory effect of pure compounds, alone and in combination (peak 2 and the combination of peak 1 and peak 2) on colon cancer cells (LoVo). These compounds were isolated from Deschampsia antarctica extracts as described in Example 3 above.

The inhibitory effect on cellular proliferation was observed with peak 2 and with a combination of peak 1 and 2 at concentrations of 1.7 mM, which correspond to 1000 μg/ml, this concentration being 10 times higher than the inhibitory concentration of the ethyl acetate and methanol extracts. This result proves that methanol and ethyl acetate extracts of Deschampsia antarctica are efficient in 10 times lower concentration than the purified compounds.

### EXAMPLE 5

### Preparation of Fast-Dissolving Tablets for Oral Administration Comprising 500 mg of Despchampsia antarcica Extract

We provide here a composition for oral administration of the Deschampsia antarcica extract for prophylactic, preventive and curing purposes for patients suffering or prone to cancerous and tumoral diseases. Tablets each exhibiting the following qualitative and quantitative composition:

Deschampsia antarctica extract 500 mg, D-glucosa monohydrate 597.6 mg, Sodium croscarmellose 35.2 mg, Microcrystaline cellulose 160.0 mg, Anhydrous citric acid 35.2 mg, Granulated sorbitol 160.0 mg, Aspartame 28.8 mg, Saccharin sodium 14.4 mg, Glycerol dibehenate 16.0 mg, Magnesium stearate 6.4 mg, Orange flavoring 46.4 mg, are preparared in the following way: all the components, with the exception of lubricating agents (magnesium stearate and glycerol dibehenate), are mixed by means of a tumbler until a homogeneous whole is obtained, the magnesium stearate and glycerol dibehenate are added and mixing is again carried out until homogeneous, then the resulting mixture is subjected to tableting in order to obtain tablets exhibiting a unit weight 1.6 g which measure 20 mm in diameter and 4.5 in height. The tablets thus prepared disintegrate in the mouth in 30 seconds.

### EXAMPLE 6

### Preparation of Fast-Dissolving Tablets Comprising 7.5 g of Deschampsia antarctica Extract

Tablets exhibiting the following qualitative and quantitative composition for 100 g:

Ingredients Quantity: Deschampsia antarctica extract 7.5 g, Spray-dried mannitol 71.0 g, Microcrystalline cellulose 15.0 g, Sodium croscarmellose 3.0 g, Ammonium glycyrrhizinate 0.3 g, Aspartame 1.0 g, L-menthol 0.2 g, Mint flavouring 1.0 g, Magnesium stearate 1.0 g are prepared in the following way: all the components, with the exception of Magnesium stearate, are mixed by a tumbler until a homogeneous whole is obtained, the Magnesium stearate is added and mixing again carried out until homogenous, then the mixture is subjected to tableting. The tablets thus prepared disintegrate in the mouth in 20 seconds.

### EXAMPLE 7

### Preparation of Pellets Containing Deschampsia antarctica Extract

900 g of Deschampsia antarctica extract, 800 g of microcrystalline cellulose, 12 g of colloidal silicon dioxide, 684 g of sodium chloride and 36 g of potassium chloride were mixed. The mixture was transferred to a fluidization rotogranulator, and a mixture of 40 g of 35% dimethyl polysiloxane emulsion and 2000 ml of ion-exchanged water was sprayed onto it. Spraying speed of the pelletizing liquid was set at 50 ml/min, pressure of the spraying air was 2.5 bar. The speed of the rotor was set at 450 rev/min in the first 15 minutes of the pelletization and later kept at 600 rev/min. Speed by volume of the fluidization air was kept at 60 m3/hour in the first 15 minutes of the pelletization and later at 90 m³/hour. The temperature of the fluidization air was set at 25° C. in the first part of the pelletization and 40° C. for the drying procedure. The dried pellets were passed through sieves 1.6 mm.

1. An antineoplastic extract, said extract being prepared from Deschampsia antarctica plants.

2. The extract of claim 1, wherein the plants are grown in vitro and treated with a physical or chemical process increasing polyphenol content of plant tissues.

3. The extract according to claim 2, wherein the physical process is UV irradiation.

4. The extract according to claim 3, wherein 45-75 μW/UV irradiation cm² is provided to the plants for 2 hours.

5. The extract according to claim 2, wherein the chemical process is incubation in salt solution.

6. The extract according to claim 5, wherein the salt, solution is 2-4M NaCl.

7. The extract according to claim 6, wherein the plants are soaked in the solution for 30 minutes.

8. An antineoplastic composition comprising at least one purified main component of the extract of claim 1.

9. The antineoplastic composition of claim 8, wherein the main component is selected from the group consisting of isoswertiajaponin ((7-O-methylorientin) 2″-O-beta-arabinopyranoside) and orientin 2″-beta-arabinopyranoside.

10. A method to prevent proliferation of cancer cells by treating patients with the extract of claim 1.

11. A method to prevent proliferation of cancer cells by treating patients with the extract of claim 2.

12. A method to prevent proliferation of cancer cells by treating patients with the composition of claim 8.

13. A method to prevent proliferation of cancer cells by treating patients with the composition of claim 9.

14. The method of claim 10, wherein the extract is administered orally to the patients.

15. The method of claim 10, wherein the cells are colon cancer cells.

16. The method of claim 10, wherein the cells are hepatoma cells.

17. A composition having antineoplastic activity, said composition comprising the extract of claim 1.

18. A composition having antineoplastic activity, said composition comprising the extract of claim 2.

19. The composition of claim 17, wherein the composition is in form of a tablet and the tablet comprises 500 mg of the extract.

20. The composition of claim 19, wherein the tablet comprises 500 mg of the extract, D-glucose monohydrate 597.6 mg, Sodium croscarmellose 35.2 mg, Microcrystaline cellulose 160.0 mg, Anhydrous citric acid 35.2 mg, Granulated sorbitol 160.0 mg, Aspartame 28.8 mg, Saccharin sodium 14.4 mg, Glycerol dibehenate 16.0 mg, Magnesium stearate 6.4 mg, and Orange flavoring 46.4 mg.

21. The composition of claim 17, wherein the composition is in form of a tablet and the tablet comprises 7.5 g of the extract.

22. The composition of claim 21, wherein the tablet comprises 7.5 g of the extract, Spray-dried mannitol 71.0 g, Microcrystalline cellulose 15.0 g, Sodium croscarmellose 3.0 g, Ammonium glycyrrhizinate 0.3 g, Aspartame 1.0 g, L-menthol 0.2 g, Mint flavouring 1.0 g, and Magnesium stearate 1.0 g.

23. The composition of claim 17, wherein the composition is in a form of a pellet, ant the pellet comprises 900 g of the extract.

24. The composition of claim 23, wherein the pellet comprises 900 g of the extract, 800 g of microcrystalline cellulose, 12 g of colloidal silicon dioxide, 684 g of sodium chloride and 36 g of potassium chloride.
